(19) United States
(12) Patent Application Publication
(10) Pub. No.: US 2015/0094744 A1
(43) Pub. Date: Apr. 2, 2015

(54) VASCULAR ANASTOMOSIS STENT

(71) Applicants: H. LEE MOFFITT CANCER CENTER AND RESEARCH INSTITUTE, INC., Tampa, FL (US); UNIVERSITY OF SOUTH FLORIDA, Tampa, FL (US)

(72) Inventors: Kamran Aghayev, Tampa, FL (US); Frank Vrionis, Tampa, FL (US)

(21) Appl. No.: 14/402,351

(22) PCT Filed: May 28, 2013
(86) PCT No.: PCT/US2013/042898

§ 371 Date: Nov. 20, 2014

Related U.S. Application Data
(60) Provisional application No. 61/651,618, filed on May 25, 2012.

(51) Int. Cl. A61B 17/11

(52) U.S. Cl. CPC A61B 17/11; A61B 2017/1107; A61B 2017/1132
USPC 606/153

(57) ABSTRACT

Provided herein are devices, methods, and kits for a stent based anastomosis.

### CROSS-REFERENCE TO RELATED APPLICATIONS

This application claims benefit of U.S. Provisional Application No. 61/651,618, filed May 25, 2012, which is hereby incorporated herein by reference in its entirety.

### TECHNICAL FIELD

This application relates to systems, devices, and methods for a stent-based vascular anastomotis.

### BACKGROUND

Vascular anastomosis is a commonly performed surgical procedure. However in case of small vessels, such as those in the brain where the exposed area is very small, suturing the vessel wall becomes extremely difficult. Anastomosis itself is a time-consuming and technically difficult procedure. Therefore simplifying anastomosis technique is extremely important, especially for brain surgery.

### SUMMARY

A vascular anastomosis stent device is disclosed for creating a vascular anastomosis without the need for suture and in less time than needed for traditional anastomosis. The stent device includes a biocompatible elongated tube structure having a first and second open end, an inside surface facing a central axis of the tube structure, and an outside surface facing away from the central axis, where the inside surface of the elongated tube is continuous between the first and second open end. The stent device also has a first radially expandable metallic ring encircling the tube structure at or near the first open end and a second radially expandable metallic ring encircling the tube structure at or near the second open end. The stent device can have additional openings as needed. For example, the stent device optionally includes a third open end such that the inside surface of the elongated tube structure is continuous between the first, second, and third open end. If the third open end is to be used to connect another blood vessel, the device can include a third radially expandable metallic ring encircling the elongated tube at or near the third open end.

The radially expandable metallic rings are optionally only partially embedded in the outside surface of the elongated tube structure without extending to the inside surface in order to reduce the risk of thrombosis. For example, the radially expandable metallic rings can be adhered to the outer surface of the elongated tube structure. In order to prevent leakage, adhesion preferably createsa water impermeable seal between the ring and the outer surface of the tube. The radially expandable metallic rings also optionally include a circumferential groove to facilitate anchoring by ring clip, band, or ligature.

In preferred embodiments, the radially expandable metallic rings are expandable by a balloon catheter. The radially expandable metallic rings optionally expand using a ratcheting mechanism that permits expansion but not collapse of the rings. However, in some embodiments, the rings are constructed from a plastically deformable metal. In either case, the rings have a radial stiffness when expanded sufficient to resist deformation when circumferential force sufficient to provide a hermetical seal between the radially expandable metallic rings and a vascular wall is applied.

The stent device can optionally include a ring clip for anchoring the vessel to the stent device. For example, the ring clip can have a first jaw and second jaw that actuate at a pivot point to define an open and closed conformation. The ring clip is preferably sized such that the jaws reside substantially within the circumferential groove when the clip is released from its open conformation. The ring clip is sized such that it is positionable over the circumferential groove and the wall of the vascular lumen when in the open conformation, but compresses the vascular wall against the radially expandable metallic ring when released from the open conformation with sufficient force to create a hermetical seal but not to deform the radially expandable metallic ring. As a non-limiting example, the ring clip can be constructed of a continuous length of wire having a coil formed at its proximal end so as to create a spring force that biases the jaws into the closed conformation.

The biocompatible elongated tube structure of the stent device may be constructed from a suitable vascular graft material. For example, the vascular graft material water is preferably water impermeable, non-thrombogenic, and elastic. Non-limiting examples of vascular graft materials that may be used include Dacron polyester, polytetrafluoroethylene (GORE-TEX®), or a combinations thereof.

The stent device is of a size and shape suitable to provide an anastomosis between blood vessels, such as those found in the human brain. Therefore, the open ends of the stent device preferably has a diameter small enough to be inserted into the lumen of the blood vessel, but large enough to allow the expandable ring to dilate the blood vessel when expanded. For example, the open ends of the stent device can be about 3 mm to about 10 mm in diameter, including about 3 mm to about 5 mm, about 3 mm to about 8 mm, and about 5 mm to about 10 mm in diameter. The elongated tube structure of the stent device can also be of any length suitable for joining blood vessels in a human, such as vessels in the brain. For example, the elongated tube structure can be about 1 cm to about 30 cm in length, including about 1 cm to about 5 cm, about 1 cm to about 10 cm, and about 3 cm to about 5 cm in length.

The stent device optionally includes a port that allows for guide wire placement, delivery of a balloon catheter, removal of air, or any combination thereof. In particular, the port has an open end and an inside and outside surface, wherein the inside surface of the port is continuous between the first and second open end.

Also provided is a method for performing a vascular anastomosis using the disclosed vascular anastomosis stent device. The method can involve inserting the stent device into a bisected vessel such that the first and second radially expandable metallic rings reside within the vessel lumen on opposing sides relative the bisection. The method can further involve expanding the first and second metallic rings to match the diameter of the lumen of the bisected vessel. Once expanded, the method can further involve applying a first ring clip, band, or ligature around the outside of the vessel at a location over the first expanded metallic ring and applying a second ring clip, band, or ligature around the outside of the vessel at a location over the second expanded metallic ring. The first and second metallic rings are optionally expanded until the lumen of the bisected vessel is fully dialated. Ring expansion is optionally accomplished using a first balloon catheter.

In preferred embodiments, blood flow is occluded proximally and distally prior to bisection. This can optionally be done using one or more aneurism clips. However, in some embodiments, blood flow is occluded using one or more of a second balloon catheter inserted beyond the point where the stent device will be inserted. The second balloon catheter can optionally be used as a guide wire for the first balloon catheter to pass over.

Once the stent device is anchored into place, the method preferably involves removing the blood flow occlusion and flushing air out of the stent device. This is preferably accomplished by allowing blood and air to spew out of the open end of the port before it is sealed, which can be accomplished using an aneurism clip.

An alternative method of performing a vascular anastomosis is provided that does not require the use of expandable rings and therefore balloon catheters. This method involves providing a vascular anastomosis stent device as described above, except that the rings do not need to be radially expandable. The method involves first serially dilating the lumen of the bisected vessel (e.g., to its maximally dilated diameter) and then inserting the stent device into the dilated lumen of the bisected vessel such that the first and second metallic rings reside within the dilated vessel lumen on opposing sides relative the bisection. The size of the stent device can be selected based on the size of the dilated lumen. The method can then involve applying a first ring clip, band, or ligature around the outside of the vessel at a location over the first metallic ring and applying a second ring clip or ligature around the outside of the vessel at a location over the second metallic ring. Blood flow can be included and air can be flushed using this method as described above.

Also provided are kits containing one or more of the above described vascular anastomosis stent devices. The kit can also contain balloon catheters, guide wires, aneurism clips, serial dilators, bands, ligatures, ring clips, or any combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an exemplary vascular anastomosis stent device.

FIG. 2 is a cross-sectional view of an exemplary ring clip in an open (FIG. 2A) and closed (FIG. 2B) conformation.

FIGS. 3A-3C are perspective views of an exemplary vascular anastomosis stent device before (FIG. 3A) and after (FIG. 3B, 3C) placement in a bisected blood vessel.

FIG. 4 is a perspective view of an exemplary T-shaped vascular anastomosis stent device.

FIGS. 5A-5B are perspective views of an exemplary T-shaped vascular anastomosis stent device connecting three blood vessel ends.

FIGS. 6A and 6B are perspective views of an exemplary radially expandable metallic ring in the collapsed (FIG. 6A) and expanded (FIG. 6B) conformation. FIG. 6C is a cross-sectional view of an exemplary vascular anastomosis stent device through the radially expandable metallic ring.

FIGS. 7A-7G illustrate an exemplary method for creating a vascular anastomosis using an exemplary vascular anastomosis device.

FIGS. 8A-8F illustrate an exemplary method for creating a vascular anastomosis using an exemplary vascular anastomosis device.

FIGS. 9A-9E illustrate an exemplary method for creating a vascular anastomosis using an exemplary vascular anastomosis device and a serial dilator.

### DETAILED DESCRIPTION

Implementations of the present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

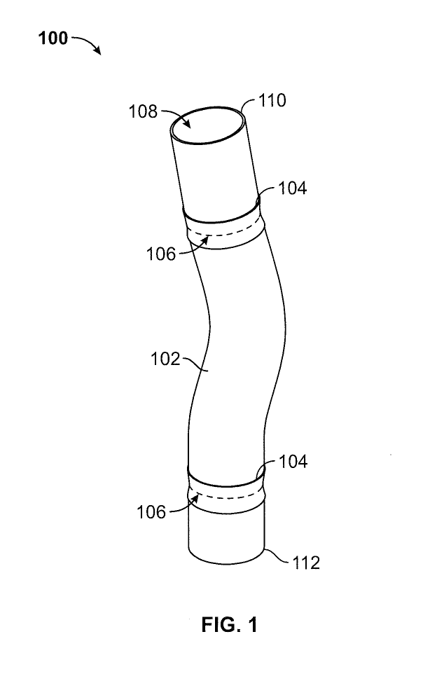

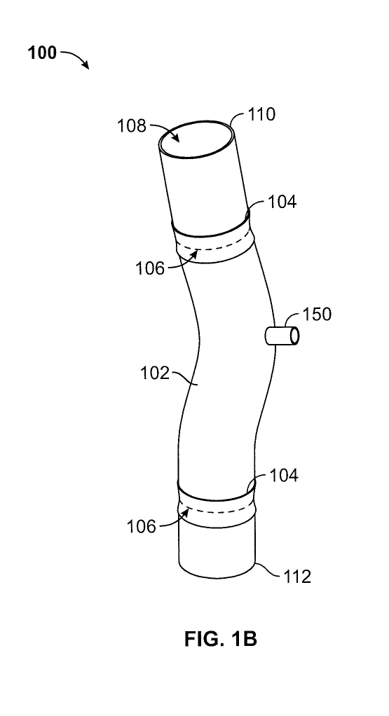

FIG. 1 is a perspective view of an exemplary embodiment of a vascular anastomosis stent device 100. The stent device 100 is an elongated tube structure 102 having an inner and outer surface defining a lumen 108 and having a first open end 110 and second open end 112.

The stent device 100 also includes radially expandable metallic rings 104 encircling the tube structure 102 positioned at or near the open ends 110,112 for anchoring the stent device 100 in the vessel lumen. In particular, the rings 104 can be positioned up to 3 mm away from the open ends 110,112, including about 0 mm, 0.5 mm, 1.0 mm, 1.5 mm, 2.0 mm, 2.5 mm, or 3.0 mm away from the open ends 110,112. In a preferred embodiment, the rings 104 are positioned about 1 mm away from the open ends 110,112.

The metallic rings 104 are optionally only partially embedded in the outside surface of the elongated tube structure and do not extend to the inside surface. The metallic ring 104 optionally has a circumferential groove 106 on the outer surface. Alternatively, the stent device can contain two rings 104 spaced apart so as to create a groove 106 between them. In some embodiments, the expandable metallic ring 104 has a suture ring on its outer surface so that it may be sutured to the vessel wall after expansion.

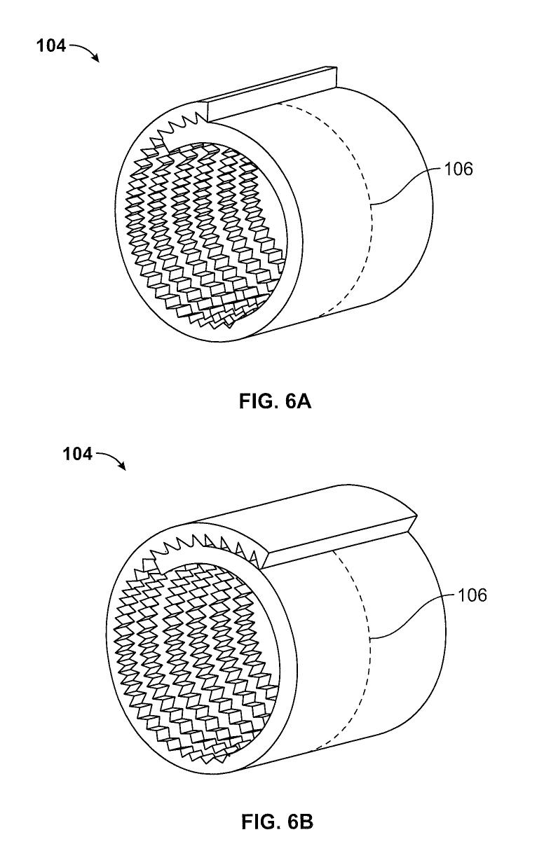

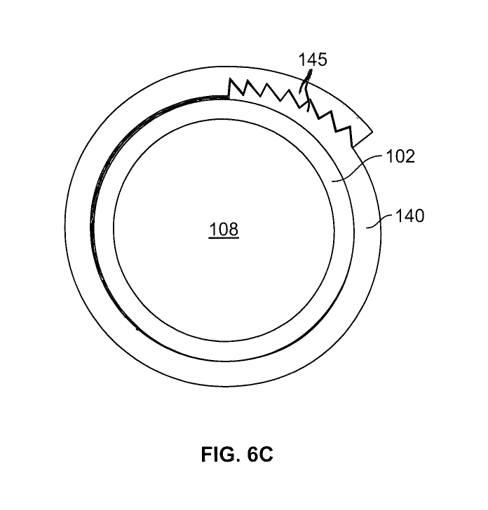

In some embodiments, the radially expandable metallic ring 104 has a ratcheting mechanism for radial expansion without contraction. For example, FIGS. 6A and 6B demonstrate a simple ratcheting expansion ring in the collapsed (FIG. 6A) and expanded (FIG. 6B) configurations. The ring 104 is preferably adjustable to multiple expanded configurations depending on the size of the vessel lumen. For example, one end of the ring 104 can contain a plurality of teeth 145 shaped, aligned, and positioned to mate with one or more teeth 145 on the opposite end of the ring 104 such that expansion of the ring results in ratcheting of the plurality of teeth 145 shaped across the one or more teeth 145. The teeth 145 may be staggered as shown in FIGS. 6A and 6B to provide the maximum number of locking positions over the expansion distance without sacrificing radial stiffness.

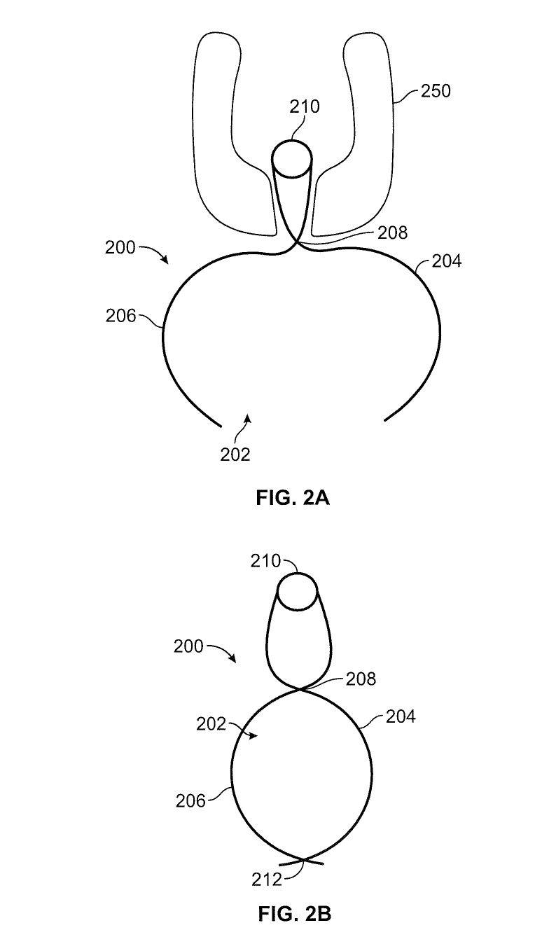

Referring now to FIGS. 2A and 2B, a ring clip 200 for securing blood vessel walls over the ends of the stent device 100 are shown. The ring clip 200 can have a first jaw 204 and second jaw 205 that actuate at a pivot point 208 to define an open (FIG. 2A) and closed (FIG. 2B) conformation. The ring clip 200 can be constructed of a single continuous length of wire to form a coil 210 at the proximal end of the ring clip so as to create a spring force that biases the jaws 204, 205 into the closed conformation. Alternatively, the ring clip 200 can be constructed of two lengths of wire, one forming each jaw, fastened at the proximal end of the ring clip 200.

The ring clip 200 is sized so that the jaws 204, 205 can reside within the circumferential groove 106 of the expanded ring 104 when there is a vascular wall located between the clip 200 and groove 106. The jaws 204, 205 of the ring clip 200 define an inner diameter 202 that is large enough to be positionable over the circumferential groove 106 and the wall of the vascular lumen when the ring clip 200 in the open conformation, but that compresses the wall of the vascular lumen against the ring 104 in the closed conformation with sufficient force to prevent leakage but not to deform the ring 104. Optionally, the distal ends of the jaws 204, 205 contact or traverse each other at a distal junction 212 when the ring clip 200 is in the closed conformation.

Aneurysm clips, which are used to block blood flow, are designed to function like tiny coil-spring clothespin, in which the blades of the clip remain tightly closed until pressure is applied to open the blades. In some embodiments, the ring clip 300 is designed like an aneurysm clip but adapted for use in the disclosed stent device 100. For example, the straight blades of an aneurysm clip can be curved to fit around an expanded ring 104. Methods for designing and manufacturing aneurysm clips are known in the art and adaptable to the present ring clip 200. As with aneurism clips, a clip applier 250 may be used to apply the needed pressure to open the jaws and place the ring clip 200 over the expanded ring 104. In preferred embodiments, the ring clip 200 is produced using an MRI-safe metal, i.e., nonferromagnetic or weakly ferromagnetic (e.g., those made from Phynox, Elgiloy® (cobalt-chromium-nickel alloy), austentitic stainless steels, titanium alloy, or commercially pure titanium). In some embodiments, carbon fiber, ceramic, or polymethylmethacrylate (PMMA) jaws are bonded to a nonferromagnetic or weakly ferromagnetic metal spring.

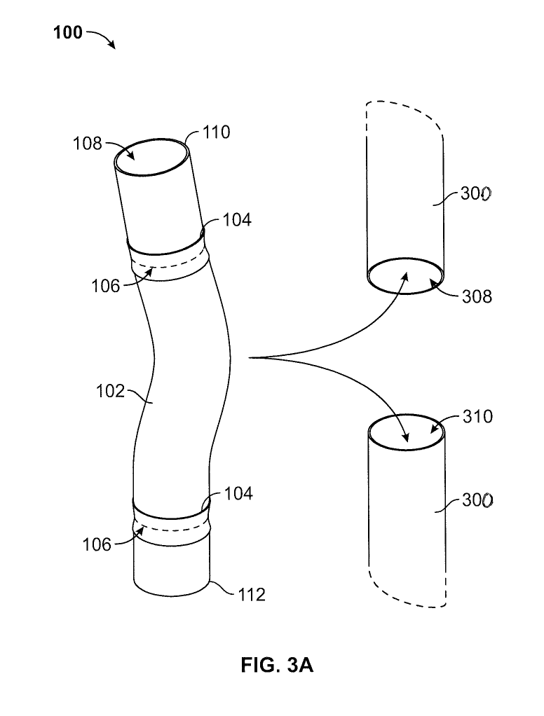

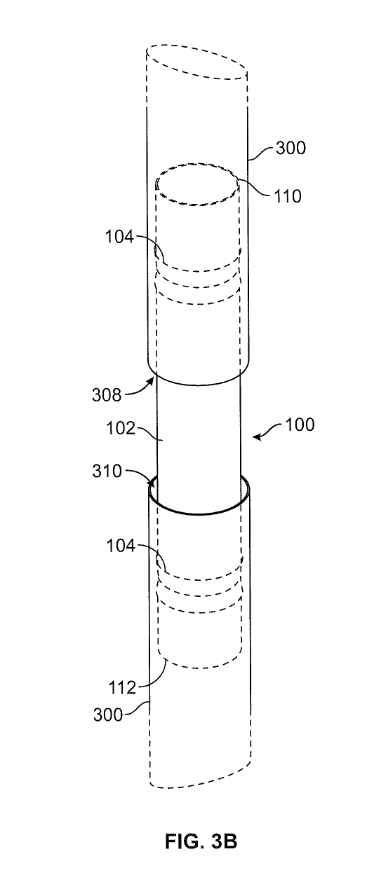

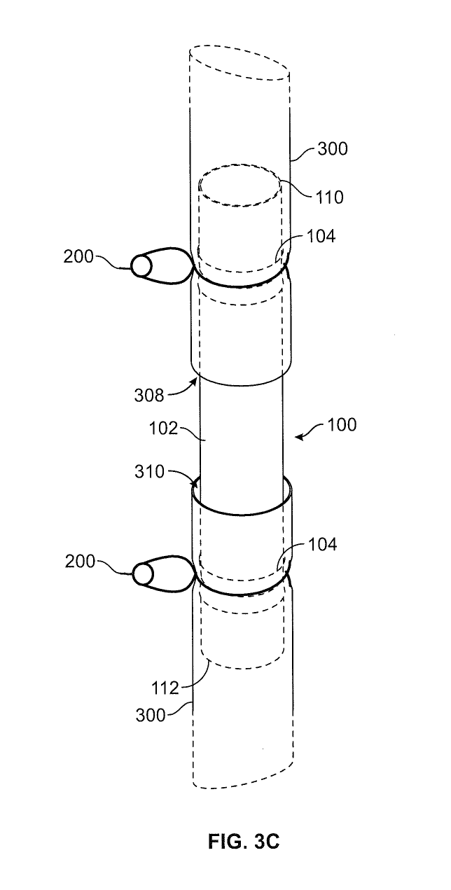

As shown in FIGS. 3A, 3B, and 3C, after a vessel 300 has been bisected, the stent device 100 can be advanced into proximal vessel lumen 308 and distal vessel lumen 310 to provide a stent-based anastomosis. Once a ring clip 200 is placed over the expanded ring 104, the anastomosis is complete. At least a portion of the elongated tube structure 102 can be exposed between the bisected vessels spanning the distance between the bisected ends.

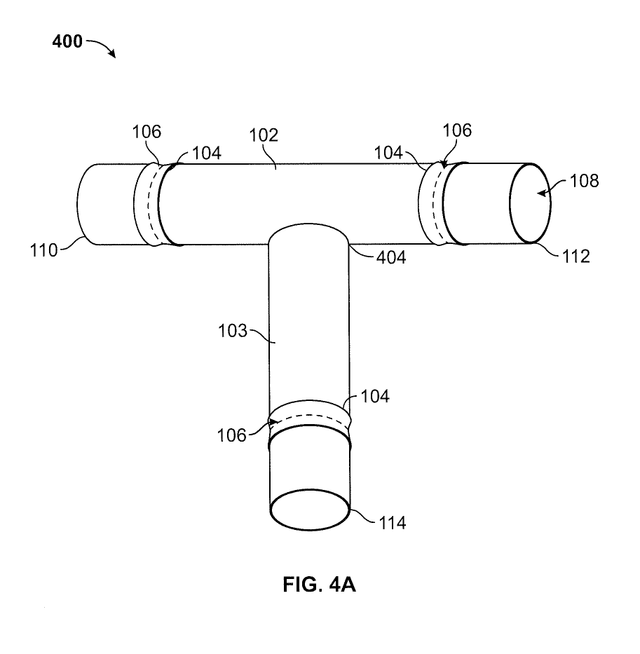

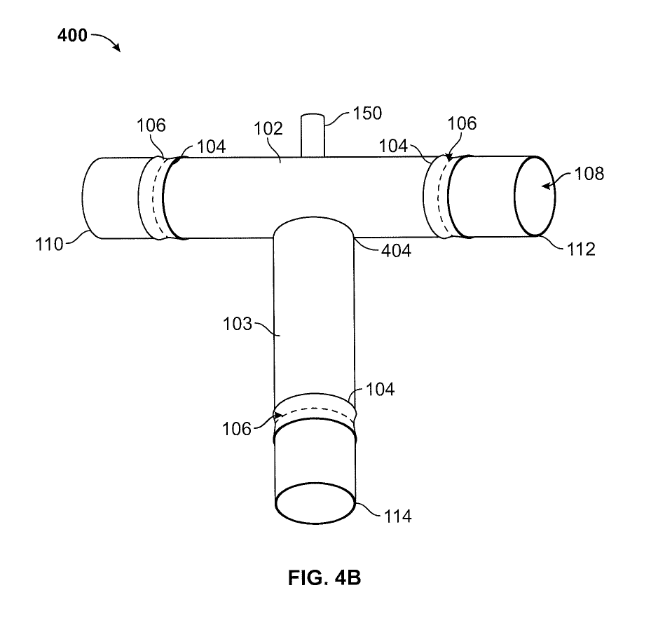

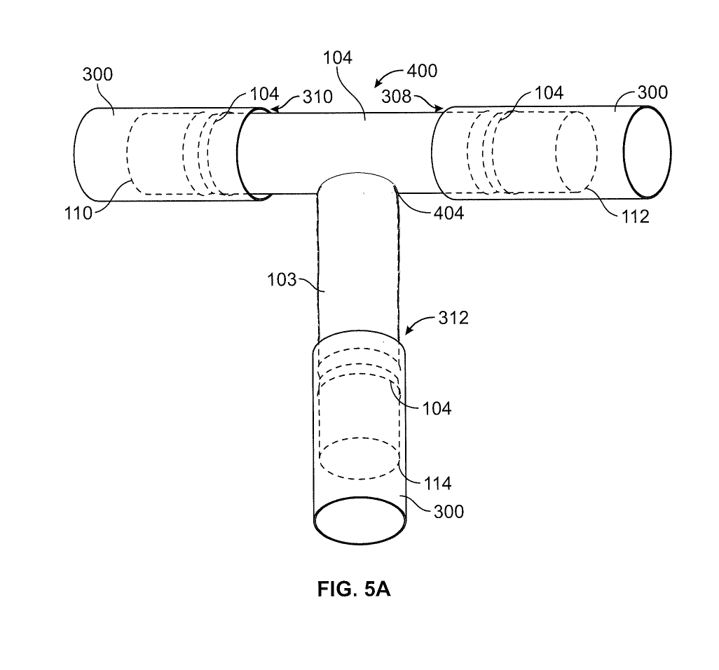

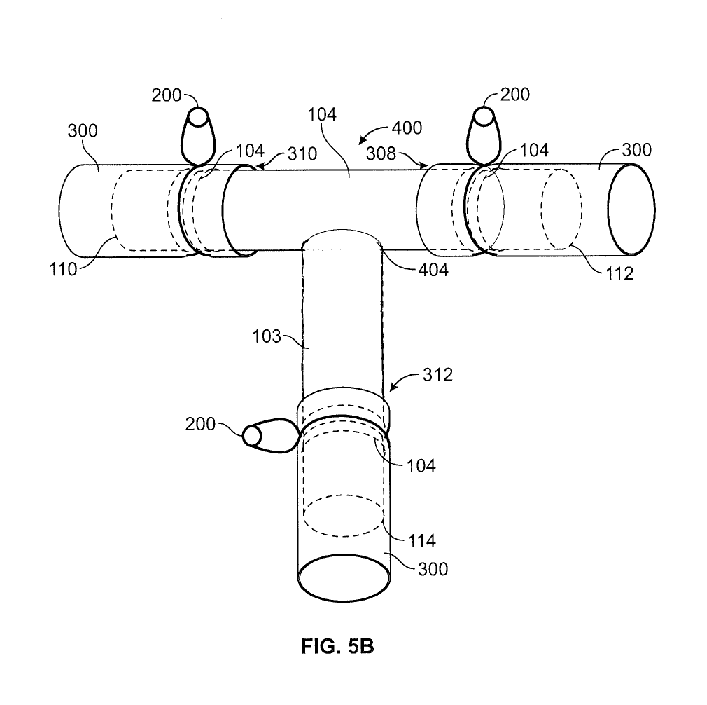

As shown in FIGS. 4A and 4B, also provided is a T-shaped stent device 400 having a first elongated tube structure 102 and a second elongated tube structure 103 joined to the first elongated tube structure 102 at an intersection 404, the elongated tube structures 102, 103 having an inner and outer surface defining a lumen 108 and having a first open end 110, a second open end 112, and a third open end 114, wherein the inside surface of the elongated tube 102, 103 is continuous between the open ends 110, 112, 114. The T-shaped stent device 400 can also have radially expandable metallic rings 104 encircling the tube structures 102, 103 positioned at or near the open ends 110, 112, 114 for anchoring. The T-shaped stent device 400 can be used where blood flow branches or to create a shunt (FIG. 5A, 5B). Other shapes for the stent, with multiple (e.g., 4, 5, 6 or more) open ends may also be designed to contain expandable rings as described herein. When there are multiple vessels being connected, it is understood that the diameter of each of the open ends can be designed based on the size of the vessels being connected and to select the desired blood flow and pressure for each vessel. Moreover, two or more stent devices may be joined together, e.g., connecting two T-shaped devices 400 together using the third open end 114 of each device.

Once in the vessel lumen, the metallic ring 104 can be palpated from outside the wall of the bisected vessel 300. In some embodiments, the expandable ring 104 is then expanded with a balloon catheter 520 to a diameter that dilates the lumen of the bisected vessel 300. Therefore, the stent device 100 can further include a port 150 for insertion of a balloon catheter. The port 150 may also be used to flush air out of the system after anastomosis is complete. Once the ring has been expanded to the desired diameter, a ring clip 200 can be applied to the vessel wall directly over each metallic ring 104. In some embodiments, e.g., small vessels, a ligature or band can be used instead of a ring clip 200. This results in compression of vascular wall between the clip 200 (band or ligature) and ring 104 providing hermetic isolation. In some embodiments, the vessel is sutured to the ring, e.g., via the optional suture ring discussed above.

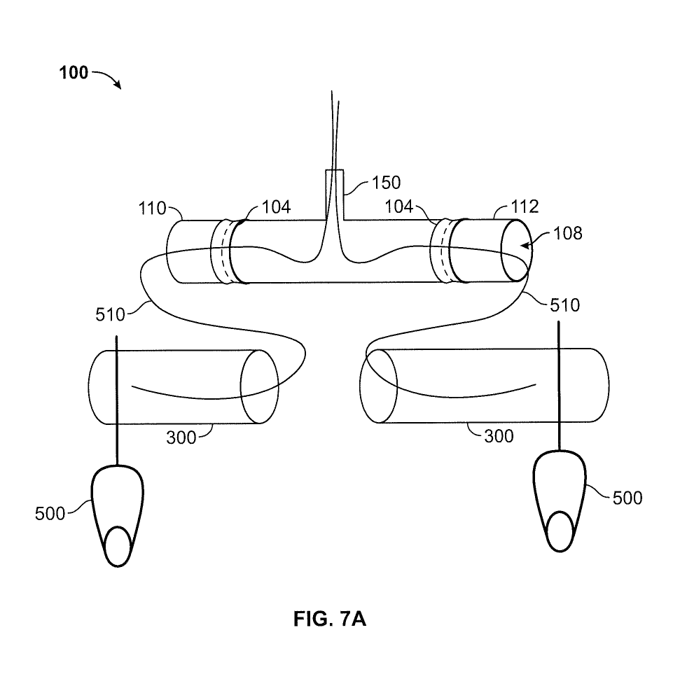

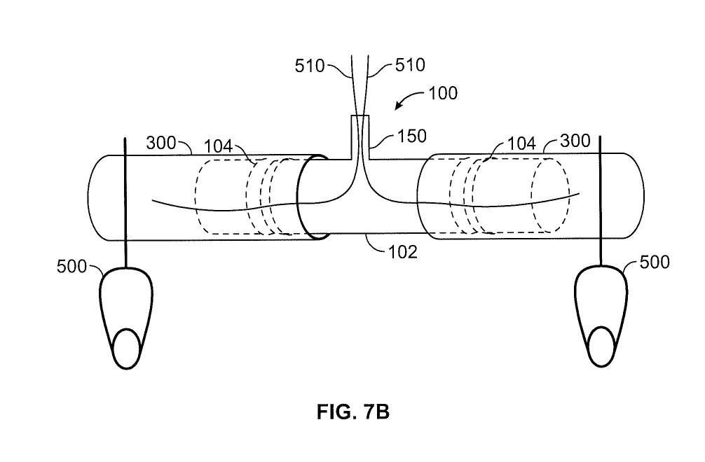

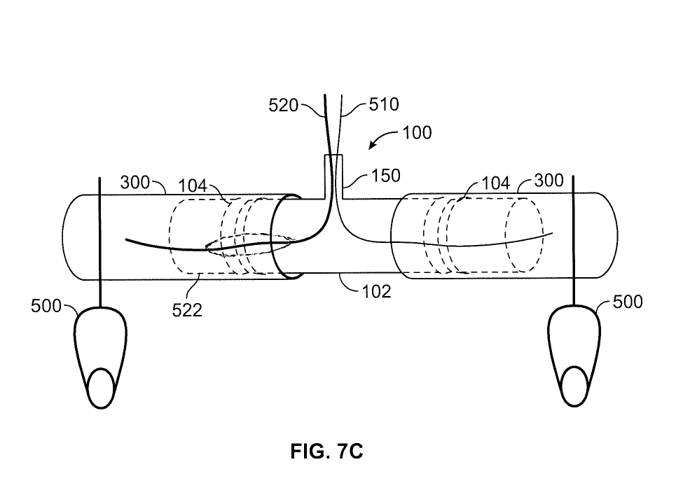

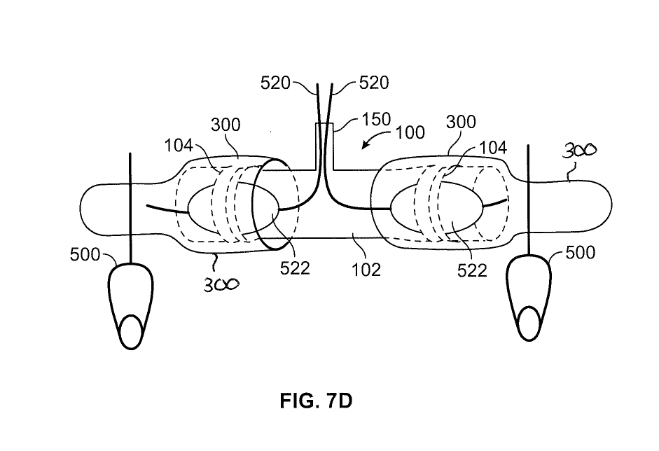

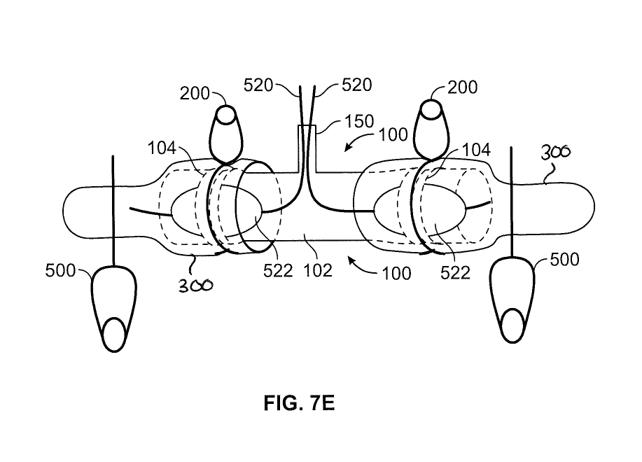

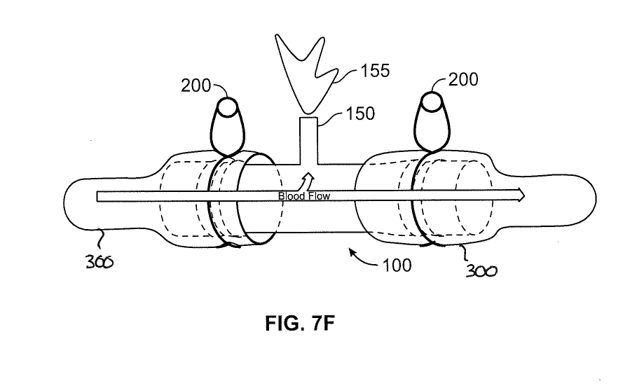

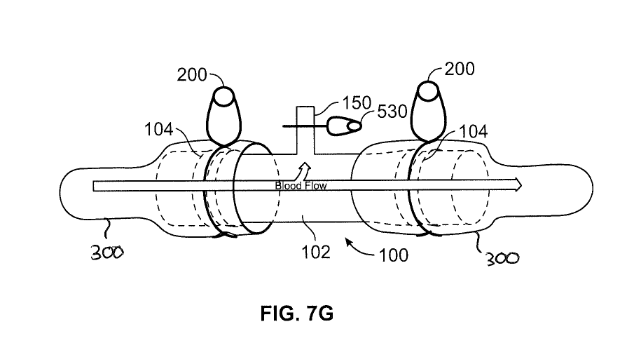

FIGS. 7A to 7G illustrate a first exemplary method for stent-based anastomosis using the disclosed stent device 100. In this method, aneurism clips 500 are used to stop proximal and distal blood flow before the blood vessel is bisected. Two guide wires 510 are inserted through the port 150, through the open ends 110, 112 and into the lumen of the proximal and distal ends of the blood vessel 300. The guide wires 510 facilitate placement of the stent device 100 (FIG. 7B). In addition, a balloon catheter 520 can be fed over the guide wire to position the balloon 522 inside the expandable ring 104. The balloon 522 is then inflated to expand the ring 104 to a diameter that dilates the lumen of the bisected vessel 300 (FIG. 7D). Once both rings 104 are expanded, ring clips 200 are applied to the vessel wall directly over each metallic ring 104. Alternatively, a band or ligature may be used, e.g., when the vessel is small. The ring clip 200 may also be sutured to the expanded ring 104, e.g., via the suture ring discussed above. The balloon catheter and guide wire may be removed when the ring clips are secured. The aneurism clips may then be removed to allow blood flow to resume. Blood 155 will spew out of the port 150, which will flush air out of the stent device 100. Air can also be removed by puncturing the stent or the vessel. Once air is removed, the port 150 is sealed. As shown in FIG. 7G, a port clip 540 can be placed over the port 150 to stop blood flow. In some embodiments, the material of the port 150 is heated and compressed to form a heat seal. In still other embodiments, a ligature is placed around the port and tightened with a surgical knot.

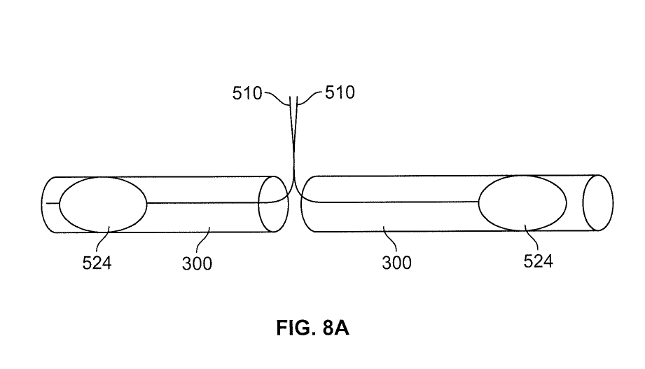

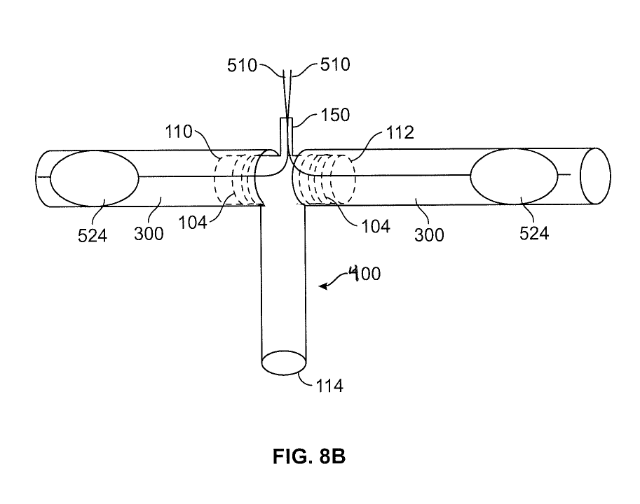

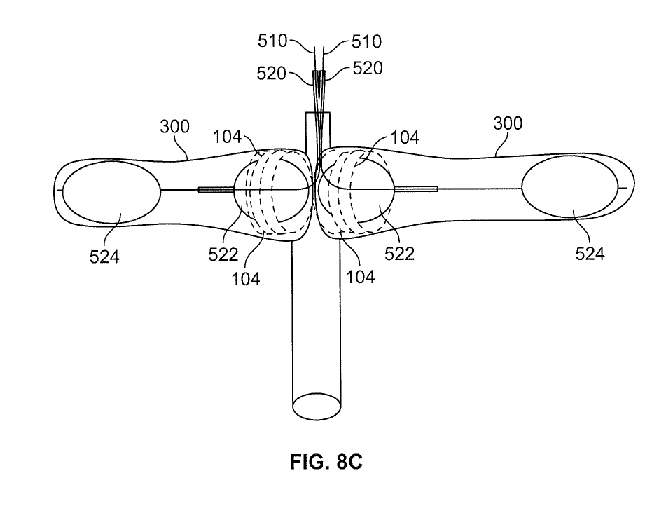

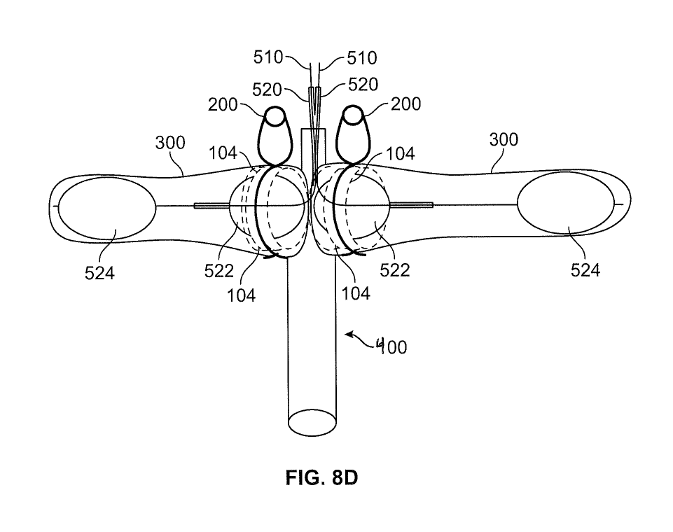

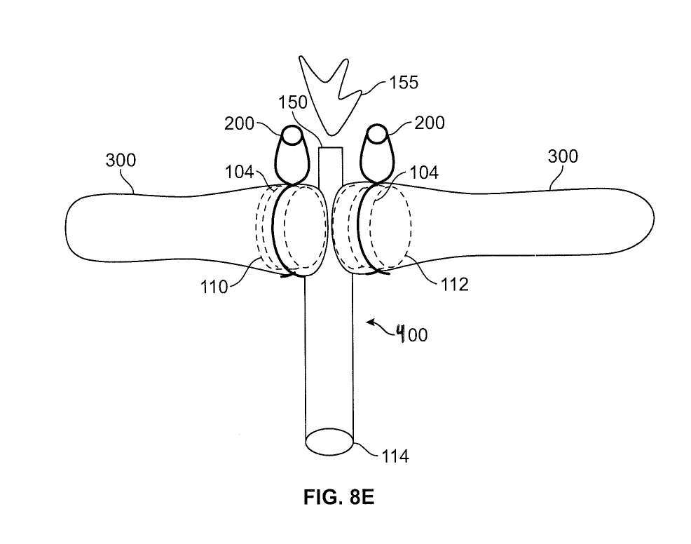

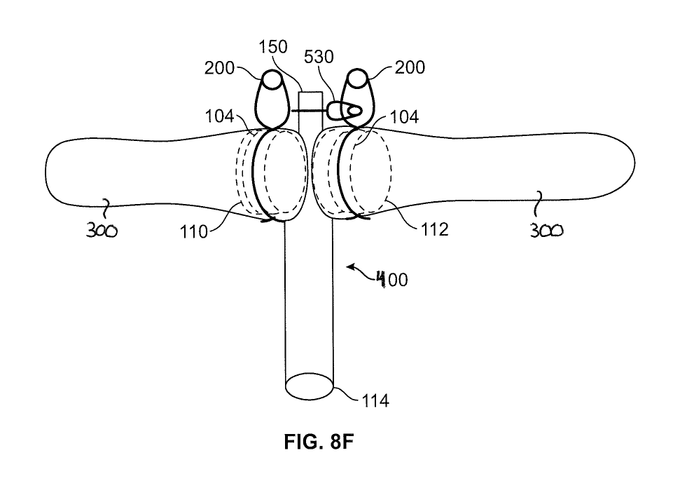

FIGS. 8A to 8F illustrate a second exemplary method for stent-based anastomosis using the disclosed stent device 100. This method involves the use of a double balloon catheter—one to stop blood flow and the other to expand the ring 104. This method is particularly useful whether the exposed area is very small. In this method, as above, aneurism clips 500 are temporarily used to stop proximal and distal blood flow before the blood vessel is bisected. The aneurism clips are slightly opened long enough to insert a balloon catheter/guide wire 510 into the lumen of the proximal and distal ends of the blood vessel 300. The guide wire balloon 524 is inflated to stop blood flow, allowing the aneurism clips 500 to be removed (FIG. 8A). The balloon catheter/guide wire 510 is then used to pass the stent device 100 into the bisected vessel 300 (FIG. 8B). A second balloon catheter 520 is guided over the balloon catheter/guide wire 510 through the port 150, through the open ends 110, 112 and into the lumen of the proximal and distal ends of the blood vessel 300. As above, the balloon 522 is then inflated to expand the ring 104 to a diameter that dilates the lumen of the bisected vessel 300 (FIG. 8C). Once both rings 104 are expanded, ring clips 200 are applied to the vessel wall directly over each metallic ring 104 (FIG. 8D). The balloon catheter 520 and balloon guide wire 510 may be removed when the ring clips are secured. Blood 155 will spew out of the port 150 once the balloon guide wire 510 is deflated, which will flush air out of the stent device 100 (FIG. 8E). Once air is removed, the port 150 is sealed. As shown in FIG. 7G, a port clip 540 can be placed over the port 150 to stop blood flow (FIG. 8F).

The balloon catheter/guide wire 510 is then used to pass the stent device 100, 400 into the bisected vessel 300 (FIG. 8B). A second balloon catheter 520 is guided over the balloon catheter/guide wire 510 through the port 150, through the open ends 110, 112 and into the lumen of the proximal and distal ends of the blood vessel 300. As above, the balloon 522 is then inflated to expand the ring 104 to a diameter that dilates the lumen of the bisected vessel 300 (FIG. 8C). Once both rings 104 are expanded, ring clips 200 are applied to the vessel wall directly over each metallic ring 104 (FIG. 8D). The balloon catheter 520 and balloon guide wire 510 may be removed when the ring clips are secured. Blood 155 will spew out of the port 150 once the balloon guide wire 510 is deflated, which will flush air out of the stent device 100, 400 (FIG. 8E). Once air is removed, the port 150 is sealed. As shown in FIG. 7G, a port clip 540 can be placed over the port 150 to stop blood flow (FIG. 8F).

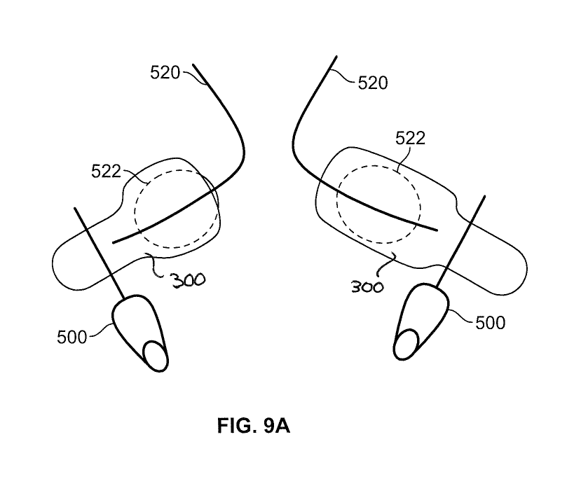

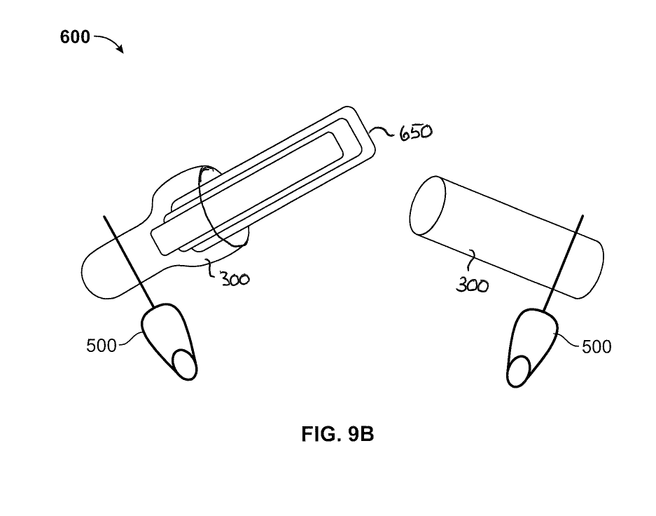

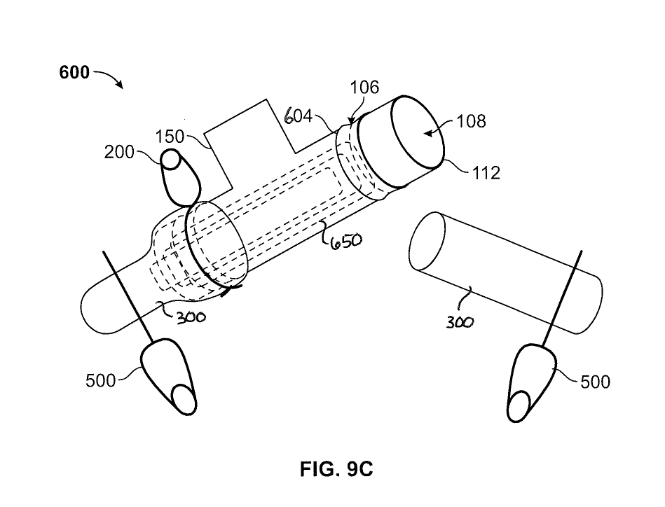

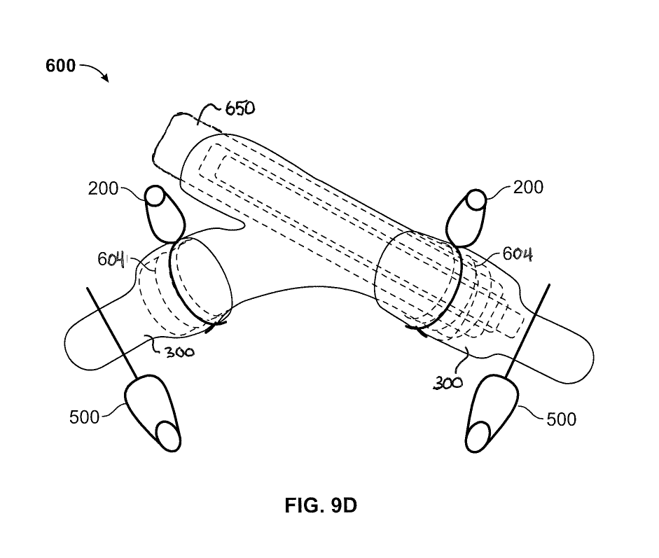

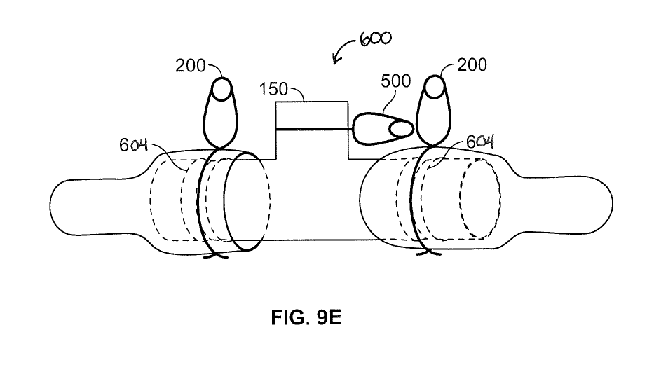

FIGS. 9A to 9E illustrate a second exemplary method for stent-based anastomosis using a variation of the stent device described above where the rings are fixed and not radially expandable 600. As above, aneurism clips 500 are temporarily used to stop proximal and distal blood flow before the blood vessel is bisected. A balloon catheter 520 can then be used to dilate the proximal and distal ends of the blood vessel 300 after bisection (FIG. 9A). A serial dilator 650 is then used to fully dilate one end blood vessel 300 and to identify the maximum diameter (FIG. 9B). The stent device 600 can then be slid along the serial dilator 650 via the first open end 110 into the dilated end of the blood vessel 300 and a ring clip 200 can then be applied to the vessel wall directly over the metallic ring 604 (FIG. 9C). The serial dilator 650 can then be used to fully dilate the opposite end of the blood vessel 300. Once dilated, the stent device 600 can then be slid along the serial dilator 650 via the second open end 112, allowing the serial dilator to pass through the port 150 (FIG. 9D). A ring clip 200 can then be applied to the vessel wall directly over the metallic ring 604 (FIG. 9D). The serial dilator 650 can then be removed through the port 150. The aneurism clips 500 may then be removed to allow blood flow to resume. Blood 155 will spew out of the port 150, which will flush air out of the stent device 100. Air can also be removed by puncturing the stent or the vessel. The port 150 can then be sealed, e.g., by placing a port clip 540 placed over the port 150 to stop blood flow (FIG. 9E).

Also disclosed is a stent device 100 having rings that are not radially expandable. In these embodiments, a serial dilator may be used to dilate the bisected blood vessel 300 to a diameter large enough to position the stent device 100 inside the blood vessel 300, e.g., by removing the largest diameter dilator and sliding the stent device 100 over the serial dilator. Once positioned inside the vessel lumen, ring clips 104 may be placed over the non-radially expandable rings to anchor the stent device in place.

The elongated tube structure 102 of the anastomosis stent device 100 can be constructed of any suitable vascular graft sheet material. The vascular graft sheet material is preferably water impermeable, non-thrombogenic, and elastic. In some embodiments, the material is a Dacron polyester, polytetrafluoroethylene, GORE-TEX polytetrafluoroethylene, or other FDA class 3 materials for implantation.

The radially expandable metallic rings 104 of the anastomosis stent device 100 (or non-radially expandable rings 604 of the stent device 600) can be constructed of any suitable MRI-safe metal, i.e., nonferromagnetic or weakly ferromagnetic (e.g., those made from Phynox, Elgiloy® (cobalt-chromium-nickel alloy), austentitic stainless steels, titanium alloy, or commercially pure titanium).

An advantage of the systems, devices and methods is simplicity of procedure. There is no need for vessel wall preparation. Usually vessel wall is cut perpendicular to axis for better approximation. Adventitia is also removed to allow accurate suturing. Also since there is no need for suturing, the systems, devices and methods result in significant reduction in anastomosis time. The vessel lumen is not exposed to sutures resulting in decreased likelihood of thrombus formation. Therefore there is no need for anticoagulation. Stent patency is maintained with anti-aggregation that has lesser morbidity.

Such a technique allows vascular anastomosis like end to end-to-end to side. Also venous anastomosis can be done easily. Usually venous anastomosis is difficult due to fragility of vessel wall. However since there is no wall suturing in this procedure, it makes the anastomosis easy.

The described systems, methods and devices are optionally used for brain revascularization; however, the systems, methods and devices can optionally used virtually in all revascularization operations. This described anastomotic technique is optionally used with a specifically designed stent that is anchored to vessel wall by applying clips or ligature outside the wall. The simplicity of the procedure allows avoiding morbidity associated with regular anastomosis.

A number of embodiments of the invention have been described. Nevertheless, it will be understood that various modifications may be made without departing from the spirit and scope of the invention. Accordingly, other aspects are within the scope of the following claims.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A vascular anastomosis stent device comprising:
a) A biocompatible elongated tube structure comprising a first and second open end, the tube structure having an inside surface facing a central axis of the tube structure and an outside surface facing away from the central axis, wherein the inside surface of the elongated tube is continuous between the first and second open end;
b) a first radially expandable metallic ring encircling the tube structure at or near the first open end; and
c) a second radially expandable metallic ring encircling the tube structure at or near the second open end.

2. The stent device of claim 1, further comprising
a) a third open end, wherein the inside surface of the elongated tube structure is continuous between the first, second, and third open end; and
b) a third radially expandable metallic ring encircling the elongated tube at or near the third open end.

3. The stent device of claim 1, wherein the radially expandable metallic rings are partially embedded in the outside surface of the elongated tube structure and do not extend to the inside surface.

4. The stent device of claim 1, wherein the radially expandable metallic rings comprise a circumferential groove.

5. The stent device of claim 1, wherein the radially expandable metallic rings comprise a ratcheting mechanism that permits expansion but not collapse of the rings.

6. The stent device of claim 1, wherein the radially expandable metallic rings comprise a plastically deformable metal.

7. The stent device of claim 1, wherein the radially expandable metallic rings are expandable by a balloon catheter.

8. The stent device of claim 1, wherein the radially expandable metallic rings have a radial stiffness when expanded sufficient to resist deformation when circumferential force sufficient to provide a hermetical seal between the radially expandable metallic rings and a vascular wall is applied.

9. The stent device of claim 1, further comprises a ring clip comprising first jaw and second jaw that actuate at a pivot point to define an open and closed conformation, wherein the ring clip is sized so that the jaws reside substantially within the circumferential groove when the clip is released from the open conformation.

10. The stent device of claim 9, wherein the ring clip is positionable over the circumferential groove and the wall of the vascular lumen in the open conformation, wherein the ring clip compresses the vascular wall against the radially expandable metallic ring when released from the open conformation with sufficient force to create a hermetical seal but not to deform the radially expandable metallic ring.

11. The stent device of claim 9, wherein the ring clip comprises a continuous length of wire having a coil formed at its proximal end so as to create a spring force that biases the jaws into the closed conformation.

12. The stent device of claim 1, wherein the biocompatible elongated tube structure comprises a vascular graft material.

13. The stent device of claim 12, wherein the vascular graft material water comprises a water impermeable, non-thrombogenic, and elastic material.

14. The stent device of claim 1, wherein the vascular graft material comprises polytetrafluoroethylene or a combination thereof.

15. The stent device of claim 1, wherein the first open end has a diameter of about 3 mm to about 100 mm.

16. The stent device of claim 1, wherein elongated tube structure has a length of about 1 cm to about 30 cm.

17. The stent device of claim 1, further comprising a port having an open end and an inside and outside surface, wherein the inside surface of the port is continuous between the first and second open end.

18. A method of performing a vascular anastomosis comprising:
a) inserting the vascular anastomosis stent device of claim 1 into a bisected vessel such that the first and second radially expandable metallic rings reside within the vessel lumen on opposing sides relative the bisection;
b) expanding the first and second metallic rings to match the diameter of the lumen of the bisected vessel;
c) applying a first ring clip or ligature around the outside of the vessel at a location over the first expanded metallic ring; and
d) applying a second ring clip or ligature around the outside of the vessel at a location over the second expanded metallic ring.

19. The method of claim 18, wherein the first and second metallic rings are expanded until the lumen of the bisected vessel is fully dialated.

20. The method of claim 18, wherein the first and second metallic rings are expanded with a first balloon catheter.

21. The method of claim 18, wherein prior to step a) the vessel is bisected by occluding blood flow proximally and distally to the site of bisection.

22. The method of claim 21, wherein blood flow is occluded with one or more aneurism clips.

23. The method of claim 21, wherein blood flow is occluded with one or more of a second balloon catheter.

24. The method of claim 23, wherein the second balloon catheter functions as a guide wire for the first balloon catheter.

25. The method of claim 21, further comprising
e) removing the blood flow occlusion;
f) flushing blood and air out of the port; and
g) sealing the port.

26. The method of claim 25, wherein the port is sealed with an aneurism clip.

27. A method of performing a vascular anastomosis comprising:
a) providing a vascular anastomosis stent device comprising
i) a biocompatible elongated tube structure comprising a first and second open end, the tube structure having an inside surface facing a central axis of the tube structure and an outside surface facing away from the central axis, wherein the inside surface of the elongated tube is continuous between the first and second open end;
ii) a first metallic ring encircling the tube structure at or near the first open end; and
iii) a second metallic ring encircling the tube structure at or near the second open end;
b) serially dilating the lumen of the bisected vessel;
c) inserting the vascular anastomosis stent device into the dilated lumen of the bisected vessel such that the first and second metallic rings reside within the dilated vessel lumen on opposing sides relative the bisection;
d) applying a first ring clip or ligature around the outside of the vessel at a location over the first metallic ring; and
e) applying a second ring clip or ligature around the outside of the vessel at a location over the second metallic ring.

28. The method of claim 27, wherein step b) comprises serially dilating the lumen of the bisected vessel to its maximally dilated diameter

29. The method of claim 27, wherein prior to step a) the vessel is bisected by occluding blood flow proximally and distally to the site of bisection.

30. The method of claim 29, wherein blood flow is occluded with aneurism clips.

31. The method of claim 29, wherein blood flow is occluded with a balloon catheter.

32. The method of claim 28, wherein the vascular anastomosis stent device further comprises a port having an open end and an inside and outside surface, wherein the inside surface of the port is continuous between the first and second open ends of the stent device.

33. The method of claim 32, further comprising
e) removing the blood flow occlusion;
f) flushing blood and air out of the stent device through the open end of the port; and
g) sealing the open end of the port.

34. The method of claim 33, wherein the port is sealed with an aneurism clip.

35. A kit comprising the vascular anastomosis stent device of claim 1 and one or more balloon catheters for expanding the radially expandable rings.

36. The kit of claim 35, further comprising one or more guide wires.

37. The kit of claim 35, further comprising one or more aneurism clips.

38. A kit comprising
a) a vascular anastomosis stent device comprising
i) a biocompatible elongated tube structure comprising a first and second open end, the tube structure having an inside surface facing a central axis of the tube structure and an outside surface facing away from the central axis, wherein the inside surface of the elongated tube is continuous between the first and second open end;
ii) a first metallic ring encircling the tube structure at or near the first open end; and
iii) a second metallic ring encircling the tube structure at or near the second open end; and
b) a serial dilator.

39. The kit of claim 38, further comprising one or more guide wires.

40. The kit of claim 38, further comprising one or more aneurism clips.
